(19) United States
(12) Patent Application Publication
(10) Pub. No.: US 2024/0363246 A1
(43) Pub. Date: Oct. 31, 2024

(54) IDENTIFICATION AND ANALYTICS OF DIAGNOSIS INDICATORS WITH NARRATIVE NOTES

(71) Applicant: Core Solutions, Inc., King of Prussia, PA (US)

(72) Inventors: Ravi Ganesan, King of Prussia, PA (US); Michael Lardieri, King of Prussia, PA (US)

(21) Appl. No.: 18/646,948

(22) Filed: Apr. 26, 2024

Related U.S. Application Data
(60) Provisional application No. 63/462,851, filed on Apr. 28, 2023.

(51) Int. Cl. G16H 50/20; G16H 10/60; G16H 50/70

(52) U.S. Cl. CPC G16H 50/20; G16H 10/60; G16H 50/70

(57) ABSTRACT

A computer implemented method for diagnosis indicator identification and analytics includes receiving a narrative note describing a first patient visit with a first patient and including unstructured data, applying a machine learning algorithm trained to use natural language processing to identify diagnosis indicators to the unstructured data to identify diagnosis indicators in the narrative note, performing at least one analysis based on information regarding the diagnosis indicators to thereby identify at least one possible patient diagnosis, and outputting a visual representation of results of the analysis for display on a display device of the end user device, wherein the result is associated with the at least one possible patient diagnosis and the visual representation facilitates clinical decision making.

### CROSS-REFERENCE TO RELATED APPLICATIONS

The present application claims priority to U.S. provisional patent application No. 63/462,851, titled Transforming Unstructured Clinical Notes into Symptoms Associated with Various Clinical Diagnoses, and Further Transforming the Symptoms into a Composite Score to Evaluate the Progress Made During Treatment and filed Apr. 28, 2023, which is hereby incorporated by reference herein in its entirety.

### TECHNICAL FIELD

The present disclosure relates generally to diagnosis indicator identification and analytics, more particularly, to machine learning methods and systems for identifying diagnosis indicators in unstructured data (e.g., clinical narrative notes) and using those indicators for behavioral health diagnostics and improved graphical interface displays.

### BACKGROUND

Within behavioral organizations, every treatment session involves capturing clinical notes. It is estimated 60%+ of data in behavioral health is unstructured notes. Conventionally, narrative notes are not used in any meaningful way. More specifically, given the voluminous and laborious nature of interpreting the notes and associating them to various symptoms, the data is often used for purposes such as supervision and billing. Given the combination of significant increase in demand for behavioral health services, advancements in machine learning and a deep understanding of the behavioral health space, a desire to interpret clinical notes has emerged within the field. Current clinicians and industry experts lack an automated way for interpreting and scoring data from clinical and case management notes. The ability to translate the notes into a feedback mechanism to measure patient progress is highly desired.

Thus, there exists a need for systems and methods that can provide an automated way of interpreting and scoring data from clinical notes and thereby increase operational efficiencies within the field of behavioral health.

### SUMMARY

Embodiments of the present disclosure address and overcome one or more of the above shortcomings and drawbacks by providing computer implemented methods for diagnosis indicator identification and analytics, a computer program product for diagnosis indicator identification and analytics, and a system for diagnosis indicator identification.

In an exemplary embodiment, a computer implemented method for diagnosis indicator identification and analytics in a data processing system having a processing device and a memory comprising instructions which are executed by the processing device is provided. The computer implemented method includes receiving from an end user device via one or more networks a narrative note having unstructured data and describing a first patient visit with a first patient applying a machine learning algorithm to the unstructured data of the narrative note to identify a plurality of diagnosis indicators, performing at least one analysis based on information regarding the plurality of diagnosis indicators to thereby identify at least one possible patient diagnosis, and outputting a visual representation of results of the at least one analysis via the one or more networks and to the end user device for display on a display device of the end user device. The machine learning model is trained at least in part to use natural language processing to identify the plurality of diagnosis indicators. The result is associated with the at least one possible patient diagnosis, and the visual representation facilitates one or more of clinical decision making and health engagement outreach.

In some embodiments, the plurality of diagnosis indicators can be one or more of a symptom or a phrase, and the phrase can describe one of a personal experience or an external experience of the first patient.

In some embodiments, the computer implemented method further includes providing to the end user device via the one or more networks the narrative note and the plurality of diagnosis indicators, obtaining from the end user device at least one indication that at least one of the plurality of diagnosis indicators are one of correct or incorrect, and retraining the machine learning algorithm based on the at least one indication.

In some embodiments, the computer implemented method further includes repeating the receiving, applying, performing, outputting, providing, obtaining, and retraining until an accuracy threshold for the machine learning algorithm is exceeded.

In some embodiments, the computer implemented method further includes deploying the machine learning algorithm on an application programming interface (API) server such that the machine learning algorithm is accessible via a provided API to a plurality of end user devices via another one or more networks.

In some embodiments, the at least one analysis includes cross-referencing each of the plurality of diagnosis indicators with a diagnosis-symptom database storing diagnoses and diagnosis indicators associated with each diagnosis, identifying at least one possible patient diagnosis for each of the plurality of diagnosis indicators based on the cross-referencing, and counting a number of occurrences for each of the at least one possible patient diagnosis.

In some embodiments, subsets of identified possible patient diagnoses with similar numbers of occurrences are clustered together in the visual representation to thereby yield insights from clusters to improve clinical decision making.

In some embodiments, the computer implemented method further includes receiving the number of occurrences for the at least one possible patient diagnosis for each of a plurality of other patients, and identifying a subset of the first patient and the plurality of other patients associated with the greatest counts for the at least one possible patient diagnosis. The visual representation includes identifiers for patients in the subset to facilitate health engagement outreach to the patients in the subset.

In some embodiments, the at least one analysis includes selecting one of the plurality of diagnosis indicators for analysis, calculating a first score for the selected diagnosis indicator based on context within the narrative note, receiving a plurality of previous scores associated with the selected diagnosis indicator and associated with a plurality of previous patient visits, and trending the plurality of previous scores and the first score for the selected diagnosis indicator. The visual representation comprises a graphical representation resulting from the trending that facilitates clinical decision making.

In some embodiments, context is one or more of an adjective or an adverb qualifying the selected diagnosis indicator.

In some embodiments, a first diagnosis indicator qualified by the adjective “severe” is scored higher than a second diagnosis indicator qualified by the adjective “mild.”

In some embodiments, the plurality of previous scores is received from a plurality of end user devices, each associated with a different health care provider for the first patient.

In some embodiments, the computer implemented method further includes cross-referencing the selected diagnosis indicator with a severity database, identifying a weight based on the cross-referencing, and calculating a weighted score by multiplying the first score for the selected diagnosis indicator by the weight.

In some embodiments, the at least one analysis includes scoring each of the plurality of diagnosis indicators based on a respective context within the narrative note, cross-referencing each of the plurality of diagnosis indicators with a severity database, identifying a respective weight for each of the plurality of diagnosis indicators based on the cross-referencing, and calculating a respective weighted score for each of the plurality of diagnosis indicators by multiplying a respective score for the plurality of diagnosis indicators by a respective weight.

In some embodiments, the at least one analysis includes normalizing and aggregating the weighted scores to produce a first composite score, receiving a plurality of previous composite scores, and trending the plurality of previous composite scores and the first composite score. The visual representation includes a graphical representation of a trend resulting from the trending that facilitates clinical decision making.

In some embodiments, the computer implemented method further includes scoring each of the plurality of diagnosis indicators based on a respective context within the narrative note, cross-referencing each of the plurality of diagnosis indicators with a severity database, identifying a respective weight for each of the plurality of diagnosis indicators based on the cross-referencing, calculating a respective weighted score for each of the plurality of diagnosis indicators by multiplying a respective score for each of the plurality of diagnosis indicators by a respective weight, normalizing and aggregating the weighted scores to produce a first composite score,
receiving a respective composite score for each of a plurality of other patients, and identifying a subset of the first patient and the plurality of other patients associated with the greatest composite scores. The visual representation comprises names of patients in the subset to assist a user of the end user device in performing health engagement outreach to the patients in the subset.

In another exemplary embodiment, a computer program product for diagnosis indicator identification and analytics is provided. The computer program product has a non-transitory computer readable storage medium having program instructions embodied therewith. The program instructions are executable by a processor to cause the processor to receive unstructured data of a narrative note describing a first patient visit with a first patient from an end user device via one or more network and an application programming interface (API) provided to the end user device, identify a plurality diagnosis indicators in the unstructured data using a machine learning algorithm trained to use one or more natural language processing algorithms, perform at least one analysis based on obtained information regarding the plurality of diagnosis indicators, and output via the one or more networks a visual representation of a result of the at least one analysis to the end user device for display.

In some embodiments, the at least one analysis includes cross-referencing each of the plurality of diagnosis indicators with a diagnosis-symptom database storing diagnoses and diagnosis indicators associated with each diagnosis, identifying at least one possible patient diagnosis for each of the plurality of diagnosis indicators based on the cross-referencing, and counting a number of occurrences for each of the at least one possible patient diagnosis. The visual representation includes a graphical representation of the at least one possible patient diagnosis with subsets of possible patient diagnoses with similar numbers of occurrences clustered together in the graphical representation. A user of the end user device can glean insights from clusters to improve clinical decision making.

In some embodiments, the at least one analysis includes selecting one of the plurality of diagnosis indicators for analysis, calculating a first score for the selected diagnosis indicator based on context within the narrative note, cross-referencing the selected diagnosis indicator with a severity database, identifying a weight based on the cross-referencing, calculating a first weighted score by multiplying the first score for the selected diagnosis indicator by the weight, receiving a plurality of previous scores associated with the selected diagnosis indicator from previous patient visits from a plurality of end user devices, each previous score being associated with a different health care provider for the first patient, and trending the plurality of previous scores and the first weighted score. The visual representation includes a graphical representation of the trend that assists a user of the end user device in clinical decision making.

In yet another exemplary embodiment, a system for diagnosis indicator identification is provided. The system has a processing device and a memory with instructions that when executed by the processing device cause the system to train a machine learning model to identify diagnosis indicators, receive a narrative note comprising unstructured data describing a first patient visit with a first patient from an end user device via one or more networks, identify using the machine learning model a plurality of diagnosis indicators in the narrative note, wherein the machine learning model is trained at least in part to use one or more natural language processing algorithms to analyze the unstructured data, output the narrative note and the plurality of diagnosis indicators to the end user device via the one or more networks, receive at least one indication that at least one of the plurality of diagnosis indicators are one of correct or incorrect from the end user device, retrain the machine learning model based on the at least one indication, determine that an accuracy threshold is exceeded for the machine learning model as a result of the retraining, and deploy the machine learning model on an application programming interface (API) server such that the machine learning model is accessible via a provided API to a plurality of end user devices via another one or more networks.

This summary is provided to introduce a selection of concepts in a simplified form that are further described below in the detailed description. This summary is not intended to identify key features or essential features of the claimed subject matter, nor is it intended to be used to limit the scope of the claimed subject matter. Additional features and advantages of the disclosed technology will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there are shown in the drawings embodiments that are presently preferred, it being understood, however, that the invention is not limited to the specific instrumentalities disclosed. Included in the drawings are the following Figures:

FIG. 1 is a flow diagram of a system for identifying and using diagnosis indicators found in a narrative note, according to an embodiment of the disclosure.

FIG. 2 is a flow chart of an exemplary method of training a machine learning algorithm to identify diagnosis indicators, according to an embodiment of the disclosure.

FIG. 3 is a flow chart of an exemplary method of diagnosis indicator identification and analytics for clinical decision support, according to an embodiment of the disclosure.

FIG. 4 illustrates a user interface into which an end user may enter a narrative note into the system, according to an embodiment of the disclosure.

FIG. 5 is an annotated narrative note showing diagnosis indicators, according to an embodiment of the disclosure.

FIG. 6 is a flowchart of an exemplary method the system can use to convert the text of a narrative note using natural language processing.

FIG. 7 is a user interface showing diagnosis indicators and associated diagnosis, according to an embodiment of the disclosure.

FIG. 8 is a clustered rubric diagram of possible patient diagnoses clustered according to diagnosis count, according to an embodiment of the disclosure.

FIG. 9 is a graph of diagnosis counts for various possible diagnoses over a period of time, according to an embodiment of the disclosure.

FIG. 10 is a flow chart of an exemplary method of diagnosis indicator identification and analytics for health engagement outreach.

FIG. 11 is a flow chart of an exemplary method of diagnosis indicator identification and analytics for clinical decision support, according to an embodiment of the disclosure.

FIGS. 12A and 12B each show a narrative note, the diagnosis indicators identified therein, and the associated scores, according to embodiments of the disclosure.

FIG. 13 illustrates the system's data module, according to an embodiment of the disclosure.

FIG. 14 is a graph of scores for a patient over a period of time, according to an embodiment of the disclosure.

FIG. 15 is a flow chart of an exemplary method of diagnosis indicator identification and analytics in clinical decision support, according to an embodiment of the disclosure.

FIG. 16 is a graph of composite scores for a patient over a period of time, according to an embodiment of the disclosure.

FIG. 17 is a flow chart of an exemplary method of diagnosis indicator identification and analytics for health engagement outreach, according to an embodiment of the disclosure.

FIG. 18 illustrates an exemplary system for diagnosis indicator identification and analytics, according to an embodiment of the disclosure.

FIG. 19 illustrates a high-level flow diagram of exemplary system operation, according to an embodiment of the disclosure.

FIG. 20 illustrates a flow diagram of system operation, according to an embodiment of the disclosure.

FIG. 21 illustrates an exemplary computing environment within which embodiment of the invention may be implemented.

FIG. 22 is a flow chart of an exemplary method of diagnosis indicator identification and analytics for clinical decision support, according to an embodiment of the disclosure.

### DETAILED DESCRIPTION

The present disclosure describes computer implemented methods, computer program products, and systems for diagnosis indicator identification and analytics. In disclosed embodiments, narrative notes describing patient interactions are analyzed by a trained machine learning algorithm to identify diagnosis indicators and analyze those diagnosis indicators in various analysis for point-of-care clinical decision support or population health tools. For example, and as will be described in greater detail below, the diagnosis indicators can be scored and trended over time or correlated to possible diagnoses that are then clustered to assist a provider in making a diagnosis and devising a treatment plan. These narrative notes cannot be converted into scores using conventional natural language processing techniques. Instead, a database that maps diagnosis indicators to clinical diagnosis is developed and used. For another example, and as will also be described in greater detail below, particular diagnosis indicators can be scored, or particular possible diagnoses counted, for a large population of patients, who can then be ranked and prioritized for outreach by providers, health insurance companies, integrated delivery systems, hospital systems, or other similar organizations.

In an example use case, a clinician completes her note at the end of a patient session, a machine learning algorithm trained to interpret natural language and to identify specific symptoms is used to identify symptoms within the note, and the identified symptoms are associated with one or more medical diagnoses, whether identified by name or by code. The machine learning algorithm can learn new words and phrases that refer to a particular symptom. This can be helpful because people may describe a symptom differently than it would be described in a medical classification list like the International Statistical Classification of Diseases and Related Health Problems, 10th revision (ICD-10). In other words, patients may not speak in the clinical terminology used in the ICD-10, but the machine learning algorithm nonetheless learns that particular words and phrases that patients use that mean the same thing as the symptom described in the ICD-10. Then, the identified symptoms are processed further to create trends, identify potential diagnoses, create scores that can be used to track progress, to identify at risk populations for outreach, and any combination of the foregoing.

With respect to scoring in particular, the present disclosure meaningfully transforms unstructured clinical notes in the mental health, substance use and intellectual and developmental disabilities specialties, for example, into symptoms associated with various clinical diagnoses and further to transforms the symptoms into a composite score to evaluate the progress made during treatment. To do this, the present disclosure uses machine learning, natural language processing and algorithms to transform data into meaningful results that can be used by clinicians and industry participants.

The ability to score the narrative notes provides clinicians quick feedback on the patient's progress without having to perform additional tests. In addition, it provides feedback to the clinicians to ensure their narrative notes are reflective of the session. With the ability to score and measure progress, other stakeholders in the organization will also be able to identify individuals who are progressing or falling behind in treatment.

The disclosed technology allows organizations to convert notes including unstructured data to symptoms, symptoms to potential diagnosis and a score to measure progress. While an individual with extensive experience may be able to use her education and experience to analyze each note and arrive at potential diagnosis, when this is done over a number of sessions each with its own notes, there is not a practical way for the clinician to look at all the data wholistically. Also, conventionally, clinicians do not score their notes. Rather, their review of their notes is more subjective and interpretive. The need to increase operational efficiencies is very critical for the success of behavioral health. The present disclosure greatly addresses the challenges of staffing shortages and high turnover, while creating better outcomes.

To describe it another way, in the field of behavior health in particular and in healthcare in general, there is a lot of information in the form of unstructured data from narrative clinical notes. Behavioral health includes mental health, substance use and intellectual disabilities. For example, unstructured data can often times include a lot of indications of a patient's illness or trends towards an illness. One problem is limited time to review every note previously prepared for a patient, e.g., over the past month. Those notes might have been written by different providers—a physician, a nurse, a home health aide-who may also be using different systems that may not be integrated with each other. It would be helpful to know what symptoms a patient is having and what diagnoses are trending, but this cannot be done with conventional natural language processing techniques.

Publications such as the American's Psychiatric Association's Diagnostic and Statistical Manual of Mental Disorders (DMS) and the World Health Organization's International Statistical Classification of Diseases and Related Health Problems (ICD) provide descriptions of the symptoms people have for particular diagnoses. These descriptions include several different symptoms that cluster together and sometimes discuss time frames required for a patient to have a symptom before it is an indicator of the diagnosis. For example, if a patient has only a particular symptom for less than twenty days, it may be too early to tell if that patient has a particular disease. The disclosed technology uses an algorithm trained to identify these symptoms. With that information, the disclosed technology can identify possible diagnoses based on those symptoms and trend the possible diagnoses, and provide all this information to a provider at the point of care so that he or she can make better decisions on the treatment for that patient.

To illustrate, the disclosed technology can also be used for measurement-based-care to see whether patients are getting better by showing if symptoms are going up or going down. Unlike the convention situation where the provider's knowledge of the patient is limited to the 20 minutes or 50 minutes session that the provider had with the patient, the provider is getting information from everyone who has touched the patient within the system of care and is therefore able to make a much better clinical decision. The provider has a better clinical picture of the patient and accordingly has an opportunity to provide better quality care.

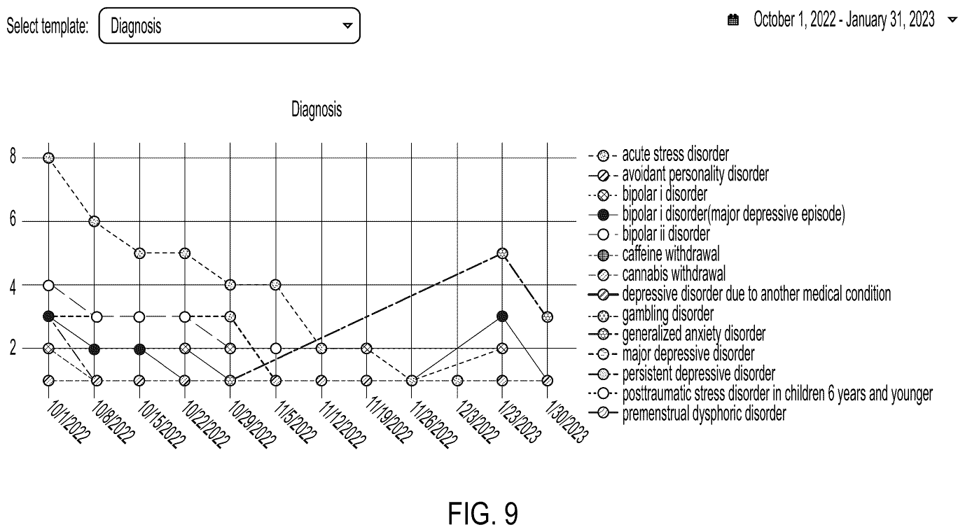

For example, a person may have had depression and anxiety three months ago, but after the patient has been treated, his or her symptomatology is likely different. Three months after treatment started, the person may have no depressive symptoms but continue to have some anxiety symptoms. The disclosed technology can generate and display charts tracking these symptoms. To illustrate, and referring to FIG. 9, one can see that the patient started out high with anxiety and depression, but depression came down and what they're dealing with right now is the anxiety.

This is important especially in the field of behavioral health because medical providers, who may be providing behavioral health treatment, may not be as attuned to behavioral health issues. They may not receive behavioral health reports. They may often receive notes that focus on the physical conditions of the patient rather than the behavioral health of the patient.

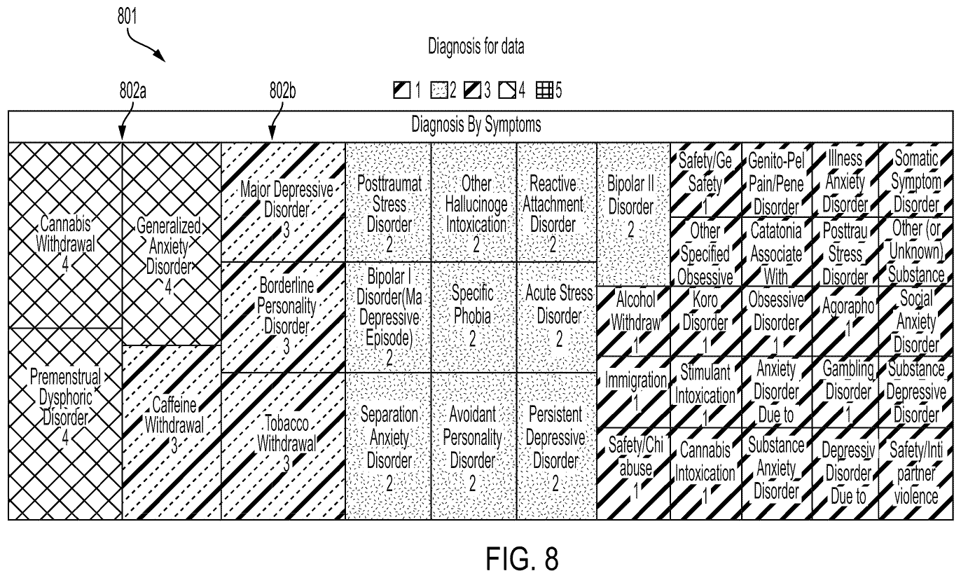

In addition to trending, the present disclosure can also be used to assist a provider in diagnosing the patient. After a patient's discussion with the provider, the provider will write or dictate a narrative note into the system. The note will be processed according to one or more of the methods described herein, e.g., method 300, and a clustered rubric diagram, such as the one shown in FIG. 8, will be generated and displayed.

The clustered rubric diagram shows visually what a provider might not necessarily pick up during a transient oral conversation or therapy session. When reviewing a narrative note in a conventional manner, a provider might think that a patient's symptoms indicating depression and anxiety. However, when reviewing the clustered rubric diagram shown in FIG. 8, the provider will see several boxes for withdrawal diagnosis clustered in the same area: cannabis withdrawal, caffeine withdrawal, and tobacco withdrawal.

This information can make a big difference to the provider. The provider just spoke to the patient who told the provider a lot of information, which the provider wrote down. The providers first inclination may have been to treat the patient for anxiety, but after reviewing the clustered rubric diagram, the provider might think, “two indicators of anxiety are caffeine withdrawal and cannabis withdrawal, which are shown on the clustered rubric diagram with large counts.” If the provider did not ask the patient about cannabis withdrawal, the provider then might decide to ask the patient additional questions before the provider recommends a treatment. For example, the provider might ask if the patient has been using cannabis and for how long has the patient not be using cannabis. Depending on the patient's responses, the provider may conclude that cannabis withdrawal is the source of the patient's anxiety and choose not to put the patient on a medication yet. Instead, the provider might suggest that the patient wait a week or two because the anxiety from cannabis withdrawal runs its course pretty quickly. The provider might plan to wait a couple of days after the cannabis is out of the patient's system before putting the patient on anxiety medication and use a non-medical approach like relaxation training in the meantime. In this way, the systems and methods described herein can be used for clinical decision support at the point of care.

The systems and methods described herein use a number of large language models to convert the unstructured data from narrative notes. Once the narrative notes are converted, symptoms are identified, and entered into a large language model (e.g., spaCy) to correlate the symptoms with diagnoses. In some embodiments, the symptoms are identified with underlining or quotation marks. The symptoms are also scored, and those scores are used on graphs for the patient.

The foregoing description focused on how the systems and methods described herein can be used for a particular patient. An end user looks at the narrative notes for an individual patient and bases the treatment plan based on the information derived from those narrative notes. However, the systems and methods described herein can also be used as a population health tool. If the system and methods described herein are used for a significant number (e.g., 500,000) of an organization's, e.g., a health insurance company, patients, the system will have a score for each of those patients. With that information, those patients can be ranked according to their scores. The organization can then have its care management staff begin to put those patients into an outreach program and begin to outreach to these patients to get them into treatment before they go to a place where they need more expensive higher cost treatment. To illustrate, if some patients have a score of 2.5 and some other patients have a score of 5.6, the organization can prioritize getting the patients with the score of 5.6 into treatment before they move on to the patients with the score of 2.5. Outreach can be done in many different ways. In some embodiments, it could take the form of a letter, a call to the patient to suggest a program that is appropriate for the patient, or an email message to the provider or care team to flag the patient for an intervention at the next appointment.

The systems and methods described herein can also be helpful for health information exchange. One patient may see several different providers. The systems and methods described herein can generate and display trends from each provider so each can have a more holistic view of how the patient's behavioral health issues are going than they otherwise would have. This information can help the provider decide what course of treatment to pursue.

The disclosed technology can have significant impacts. Forty percent of all patients have an untreated behavioral health disorder. In a population of 1,000,000 people, that means 400,000 people have some level of behavioral health disorder and are not being treated. If these patients can receive treatment, it may save health insurance companies, a lot of money. For example, assuming a savings of $2,500 a year for each patient (which is the experience of one major insurance company), and that 1,000 patients are treated, health insurance companies can save $2,500,000. Extrapolating across the entire population, even more money can be saved.

It should be noted that the disclosed technology can be implemented in a variety of applications. Accordingly, it is not intended that the disclosed technology is limited in its scope to the application set forth in the above and following descriptions or illustrated in the drawings. In particular, the presently disclosed subject matter is described in the context of use to identify, track and measure behavioral health symptoms of a patient, regardless of what system of care the patient is in, with wholistic treatment across the continuum of care no matter where the patient is currently treated as a goal. The present disclosure, however, is not so limited, and can be applicable to other applications. The present disclosure, for example and not limitation, can be useful in other fields requiring diagnosis, both medical and non-medical. Such implementations and applications are contemplated within the scope of the present disclosure. Accordingly, when the present disclosure is described in the context of the behavioral health field, it will be understood that other implementations can take the place of those referred to in any of the examples herein.

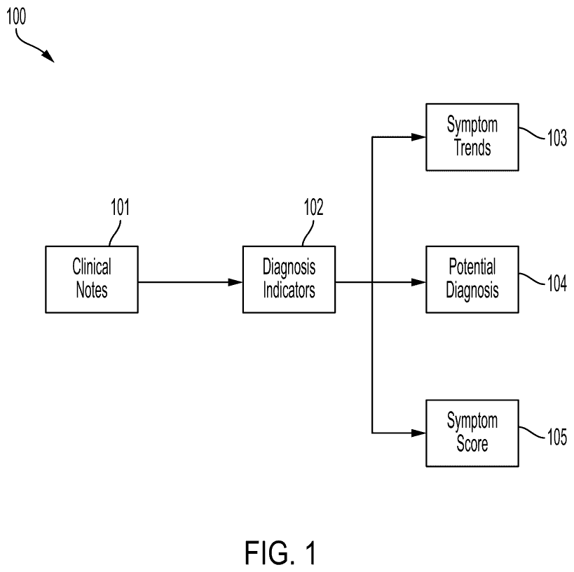

Turning now to the figures, FIG. 1 is a flow diagram of a system for identifying and using diagnosis indicators found in a narrative note, according to an embodiment of the disclosure. An end user can input a narrative note into the system 101, the system can identify diagnosis indicators in the note 102, and use the diagnosis indicators to trend the patient's symptoms 103, assist the end user in making a diagnosis 104, or calculate a score 105. The end user may be a health care provider who saw a patient for a patient visit. The provider can be any type of provider, including, for example, a nurse's aide all the way up through the disciplines to the highest level provider, i.e., physicians. The narrative note can be a clinical note describing the patient visit or a case management note or a note from a medical assistant, etc.

A diagnosis indicator is any word or phrase that may indicate, on its own or with other diagnosis indicators, a diagnosis, a disease, a health condition, or a behavior. A diagnosis indicator can be a symptom, like “irritability” for example, or a synonym of a symptom. A diagnosis indicator can also be a phrase that describes a personal experience the patient is having, whether that experience be physical, emotional, or mental. For example, “unable to control their anxious feelings” is a phrase that describes a patient's mental experience. A diagnosis indicator can also be a phrase that describes an external experience or environment to which the patient was exposed. For example, “physical abuse” is a diagnosis indicator describing an external experience a patient experiences. Unless context dictates otherwise, any reference to a “symptom” or a “synonym of a symptom” herein should be understood as referring to a “diagnosis indicator,” which may, but does not necessarily, refer to a symptom or a synonym of a symptom.

One diagnosis may have many diagnosis indicators. For example, anxiety disorder may be associated with the following diagnosis indicators: difficult to control the worry, difficulty concentrating, difficulty falling asleep, easily fatigued, feeling keyed up, feeling on edge, irritability, mind goes blank, muscle tension, person believes there is a right way of doing things and that is how things should be done even at the expense of relationships, and restlessness.

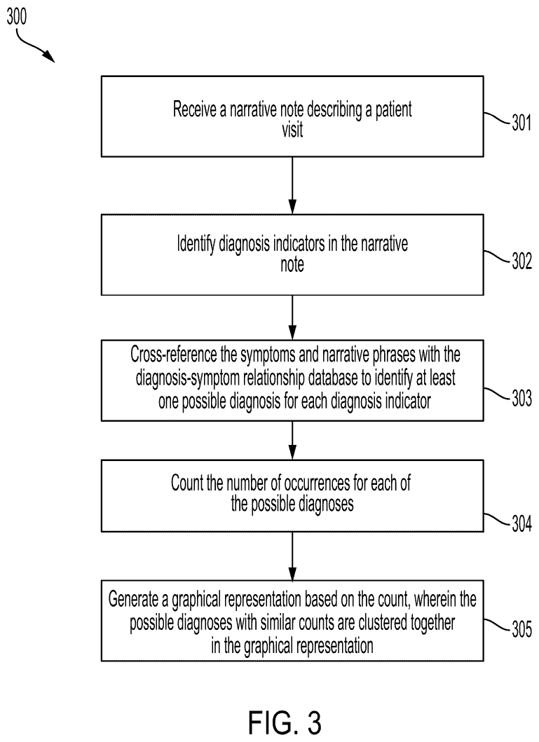

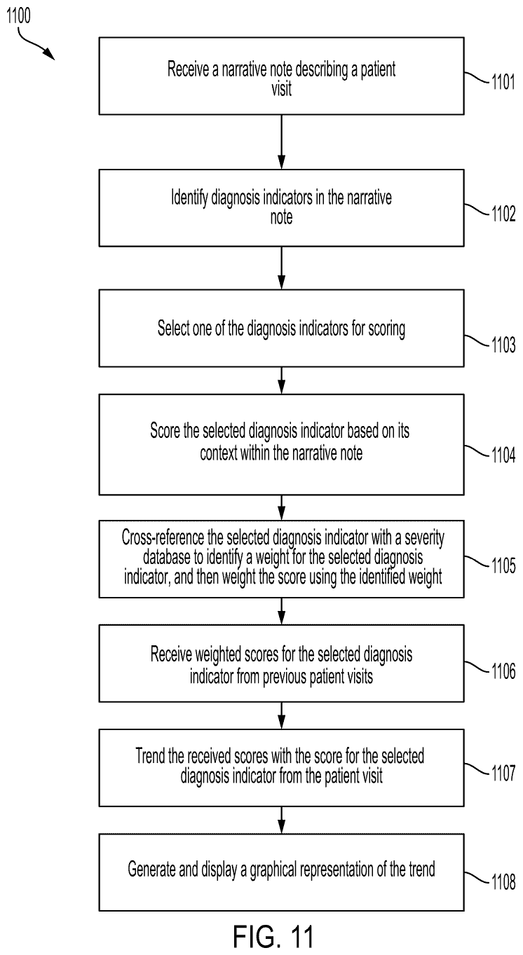

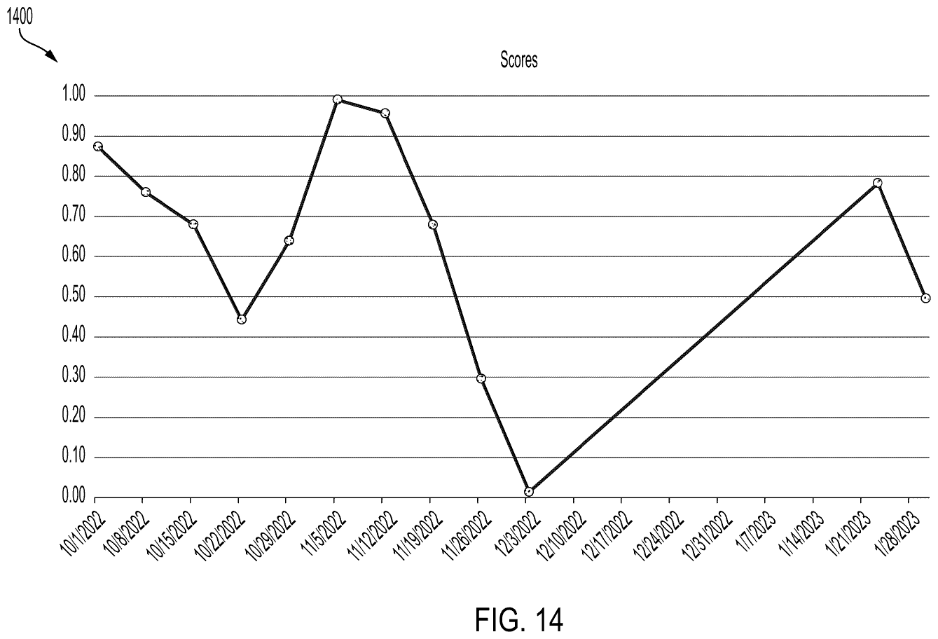

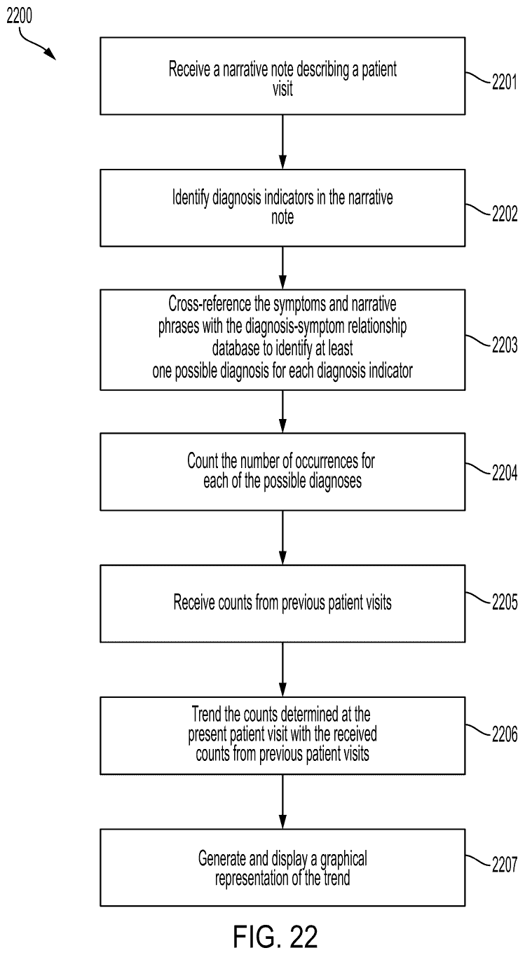

The system can trend the patient's symptoms, as described in greater detail with respect to FIGS. 22 and 11, to assist the end user in making a diagnosis, as described in greater detail with respect to FIG. 3, or to calculate a score, as described in greater detail with respect to FIGS. 11 and 14.

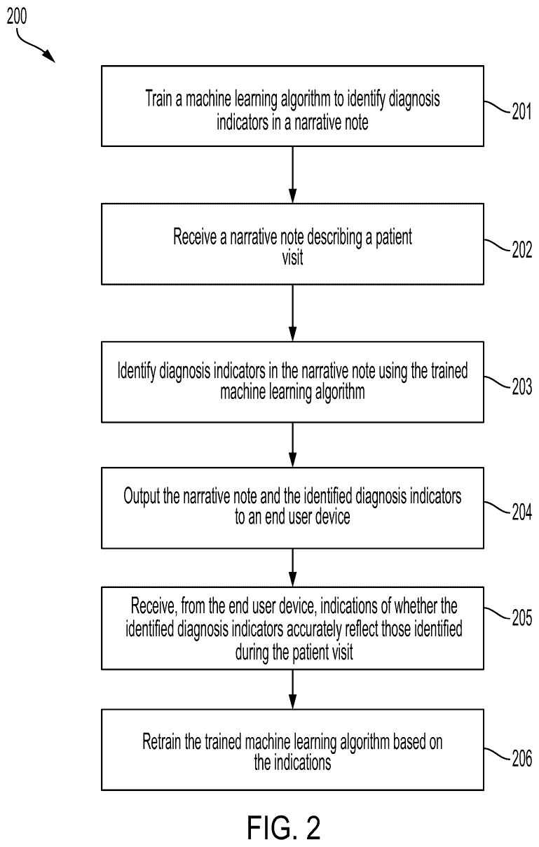

FIG. 2 is a flow chart of an exemplary method of training a machine learning algorithm to identify diagnosis indicators, according to an embodiment of the disclosure. At step 201, the method 200 can include training a machine learning algorithm to identify diagnosis indicators. At step 202, the method 200 can include receiving a narrative note describing a patient visit. At step 203, the method 200 can include using the trained machine learning algorithm to identify diagnosis indicators in the narrative note. At step 204, the method 200 can include outputting both the narrative note and the diagnosis indicators to an end user device. At step 205, the method 200 can include receiving, from the end user device, indications of whether the identified diagnosis indicators accurately reflect those identified by the user during the patient visit. At step 206, the method 200 can include retaining the trained machine learning algorithm on the indications.

This is an ongoing and iterative process in some examples: A note is written, the note processed against algorithm, the algorithm identifies exact matches for the symptom as well as synonyms that express the same concept, the provider is requested to rate the accuracy of the response which is fed back to the algorithm so it learns for the future, and another entity continues to review on the back end and add or delete symptoms and synonyms based on the findings and response from providers and the master reviewer's ongoing monitoring. This ongoing and iterative process that utilizes trained subject matter experts in evaluating the algorithm responses and determining what adaptations are needed to provide better and more accurate results over time is special and unique.

FIG. 3 is a flow chart of an exemplary method of diagnosis indicator identification and analytics for clinical decision support, according to an embodiment of the disclosure. At step 301, the method 300 can include receiving a narrative note describing a patient visit. At step 302, the method 300 can include using a trained machine learning algorithm to identify diagnosis indicators in the narrative note. In some embodiments, the machine learning model can be trained using the method described above with respect to FIG. 2. At step 303, the method 300 can include cross-referencing the diagnosis indicators identified at step 302 with the master diagnosis-symptom relationship database to identify possible diagnosis for each identified diagnosis indicator.

At step 304, the method 300 can include counting the number of occurrences for each of the possible diagnoses identified at step 303. As one of ordinary skill in the art will appreciate, many diagnoses can share multiple symptoms. For example, the symptom “anxiety” is a symptom for anxiety disorder and about ten other diagnoses in some examples. This step will show how many symptoms a patient has that match a particular diagnosis. For example, a patient may have six symptoms that match with depression, three of these same symptoms match with Bipolar Disorder as well as Borderline Personality and Cannabis Withdrawal.

At step 305, the method 300 can include generating a graphical representation based on the counts from step 304, with possible diagnoses having similar counts clustered together in the graphical representation. This graphical representation can assist an end user in clinical decision making because the end user may be able to glean insights from the clusters. With these insights, the provider may choose to further clarify and/or develop the diagnosis based on the data returned in the graphical representation. An example of a graphical representation that can be generated at step 305 is provided at FIG. 8.

The Agency for Health Research and Quality defines “clinical decision support” as providing “timely information, usually at the point of care, to help inform decisions about a patient's care. [Clinical decision support] tools and systems help clinical teams by taking over some routine tasks, warning of potential problems, or providing suggestions for the clinical team and patient to consider.” There are many forms of clinical decision support. For example, in radiology, a computer and artificial intelligence (AI) provide clinical decision support by making a recommendation to the Radiologist based on what it sees in an x-ray.

Many behavioral health disorder symptoms are present in many different diagnoses. Making a determination from a cluster of symptoms and combining them to determine a diagnosis is the art of behavioral health diagnostics. In providing a clustered rubric diagram, the disclosed technology assists the end user in making his or her clinical decision, which is not possible based on the prior art. Rendering the clustered rubric diagram showing diagnostic clusters provides this insight and will help improve diagnosis. This is one reason why the disclosed technology is so important and useful, especially in the technical field of behavioral health diagnostics.

In some embodiments, once a diagnosis is determined, the system described and illustrated herein can output treatment recommendations based on that diagnosis. The system may cross-reference the diagnosis with a database storing recommendations from various agencies, such as the Center for Disease Control, the Substance Abuse and Mental Health Services Administration, and the Health Resources and Services Administration, as well as from other evidence-based practice clearing houses. In some embodiments, the system can analyze actual data from providers and provide a treatment recommendation. For example, the system may analyze hundreds of records in a database of patient records and see that patients of a particular demographic with a particular cluster of symptoms have better outcomes when a provider pursues one treatment plan over another.

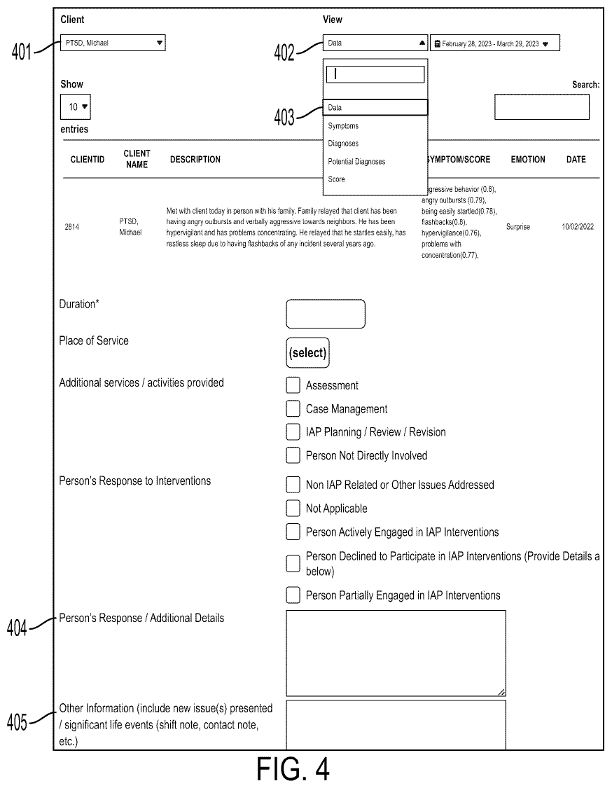

As mentioned above with respect to step 301, the method 300 can include receiving a narrative note describing a patient visit. FIG. 4 illustrates a user interface into which an end user may enter a narrative note into the system, according to an embodiment of the disclosure. The end user may be a health care provider who saw a patient for a patient visit. The end user may select the patient from the client drop down list 401 and navigate to the different modules using the view drop down list 402. With the data module 403 selected, the user interface may show the client (i.e., patient) ID, client (i.e., patient) name, a narrative note, symptoms and scores, an emotion, and the date of the visit described in the narrative note. The data module 403 may also contain data input fields that the end user can use to describe a new patient visit, including a data input field for narrative notes at 404 and 405.

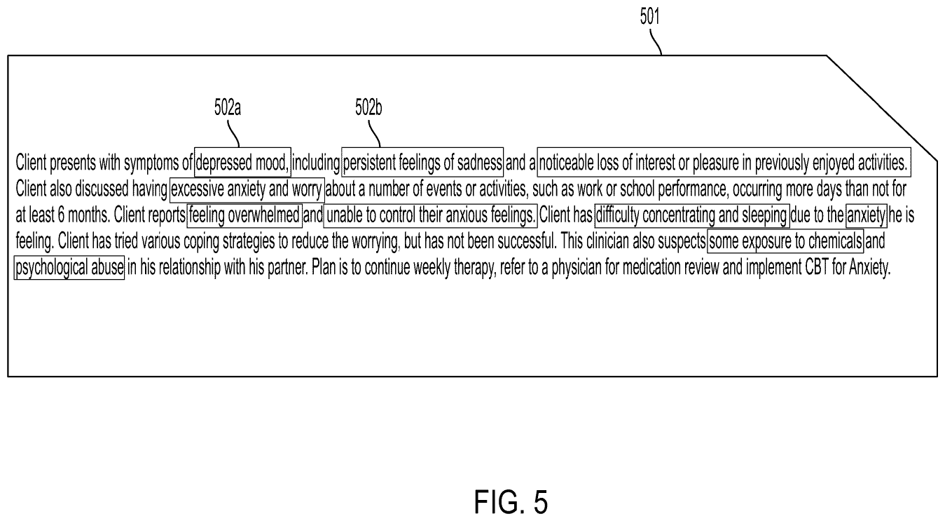

As mentioned above with respect to step 302, the method 300 can include using a trained machine learning algorithm to identify diagnosis indicators in the narrative note. FIG. 5 is an annotated narrative note showing diagnosis indicators, according to an embodiment of the disclosure. The narrative note 500 describes a provider's visit with a patient, and includes several diagnosis indicators, each shown in a box, the first two numbered as 502a and 502b. As explained above, a diagnosis indicator is any word or phrase that may indicate, on its own or with other diagnosis indicators, a diagnosis, a disease, a health condition, or a behavior. The narrative note in FIG. 5 includes several symptoms (e.g., depressed mood, feelings of sadness, anxiety, worry) and several narrative phrases (e.g., feeling overwhelmed, unable to control their anxious feelings, some exposure to chemicals, psychological abuse).

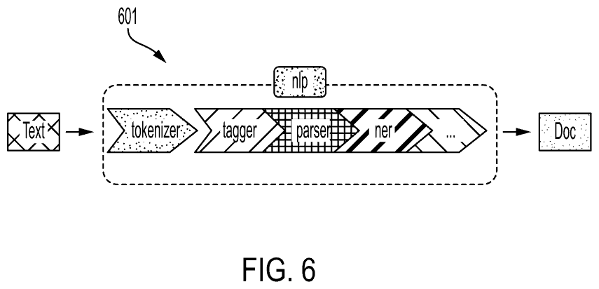

FIG. 6 is a flowchart of an exemplary method the system described herein can use to convert the text of a narrative note using natural language processing. The machine learning algorithm can use natural language processing when analyzing the narrative note to identify diagnosis indicators.

The system can use natural language processing capabilities to read narrative notes and identify symptoms. With reference to FIG. 11, the system also uses natural language processing capabilities to assign scores based on context. These capabilities are achieved by preprocessing data, converting the text, building the model, and training the model. Preprocessing can be used to convert the raw test into the clean text. In some embodiments, the following libraries are used to preprocess data: Pandas (to load the data from RDBMS to PYTHON Dataframe), Textblob (to correct the text in Dataframe of PYTHON), emoji (to read the emotions and convert it as text), and pyodbc (to connect python to database). In some embodiments, the narrative note is additionally mined to identify the emotion (e.g., happy or sad) of the patient. In some embodiments, this is done using the Tex2emotion library.

In some embodiments, natural language processing procedures, such as the one shown in FIG. 6, are used to convert the text into JavaScript Object Notation (JSON). The text is converted to tokens. In some embodiments, this is done using natural language processing-spaCy library. After the text is converted to tokens, parts of speech are predicted. In some embodiments, Tagger is used to predict part-of-speech tags for any part-of-speech tag set. Parsing is defined as the process of converting codes to machine language to analyze the correct syntax of the code.

For model building, a convolutional neural network (CNN) is used in some examples to identify symptoms. The model is first trained to identify exact symptoms and based on the learning it will identify all the symptoms in the text. CNN is a deep learning algorithm providing ˜90% accuracy in some examples.

In some embodiments, during model building, a DocBin process can be used to read the JSON file to model used to efficiently serialize the information, Tqdm can be used to create simple and efficient processing, and concise concepts can be used to get the score, although other libraries may be used to accomplish these same goals.

To train the model, the CNN algorithm is fine-tuned using simple neural networks in natural language processing to increase accuracy. In some embodiments, the Keras library is used to build a neural network.

Data is tested against multiple algorithms to find the most accurate algorithm in some examples. Further, data training can be performed on a periodic basis against multiple datasets to improve accuracy.

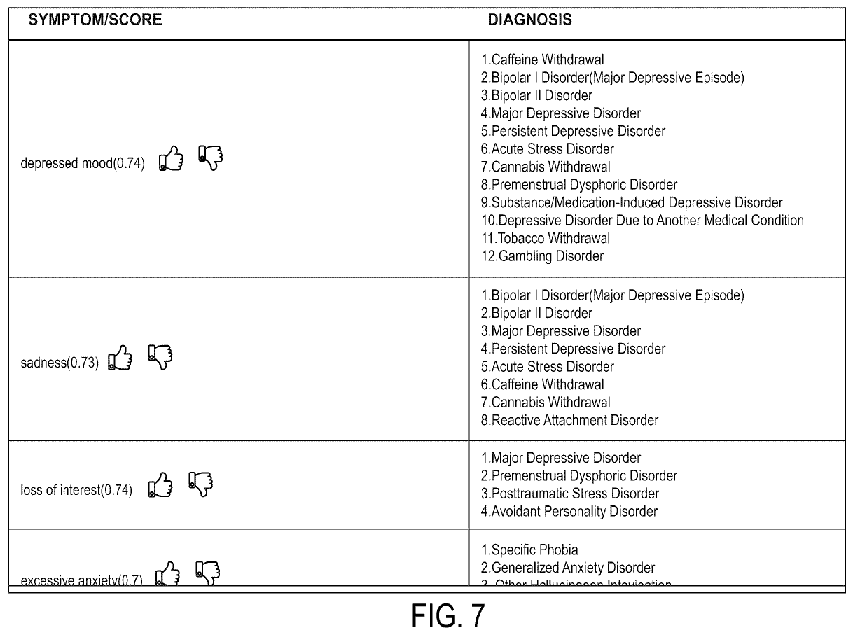

FIG. 7 is a user interface showing diagnosis indicators and associated diagnosis, according to an embodiment of the disclosure. Once the system described herein has received the narrative note and identified the diagnosis indicators within that narrative note, a screen similar to the one shown in FIG. 7 may be displayed to the end user. In some examples, the screen has two columns. The first column lists a diagnosis indicator, a score (which will be discussed in more detail with reference to FIG. 11), and feedback buttons.

The feedback buttons can be used by the end user to indicate whether or not the end user agrees that the narrative note correctly contains the diagnosis indicator. If so, the end user can select the “thumbs up” feedback button. If not, the end user can select the “thumbs down” feedback button. As described above with reference to FIG. 1, this feedback can be used to retrain the machine learning model. In this way, the machine learning model can be trained in an ongoing and iterative process to accept more synonyms that reflect the same concept based on feedback from the end user.

The second column includes all of the diagnosis for which the diagnosis indicator is an indicator. For example, a depressed mood may be an indication of various types of withdrawal, bipolar disorders, and depressive disorders, and other diagnosis.

FIG. 8 is a clustered rubric diagram of possible patient diagnoses clustered according to diagnosis count, according to an embodiment of the disclosure. Once the system has counted the number of occurrences for each of the diagnosis indicators, it can generate a graphical representation of the possible diagnoses 801, clustered according to their counts. In other words, possible diagnoses having similar counts are clustered together in the graphical representation. Further, in the embodiment shown in FIG. 8, the clusters are color-coded. One cluster 802a is comprised of cannabis withdrawal, premenstrual dysphoric disorder, and generalized anxiety disordered, which each have a count of 4. Another cluster 802b is comprised of major depressive disorder, borderline personality disorder, tobacco withdrawal, and caffeine withdrawal, which each have a count of 3.

FIG. 22 is a flow chart of an exemplary method of diagnosis indicator identification and analytics for clinical decision support, according to an embodiment of the disclosure. Steps 2201-2204 may be the same as step 304. At step 2205, the method 2200 can include receiving counts for the possible diagnoses from previous patient visits. Counts may be from the same or different providers. For example, in some embodiments, the counts may be from previous patient visits with a single provider. However, in some embodiments, the counts may be from previous patient visits with many different providers.

At step 2207, the method 2200 can include trending the counts. At step 2208, the method 2200 can include generating and displaying a graphical representation of the trend from step 2207. The graphical representation can provide a historical representation of all notes written for that patient over time by all provider types that have “touched” the patient. It can also be used by behavioral health providers and by medical providers in medical settings that would not be writing a behavioral health note. Unlike the convention situation where the provider's knowledge of the patient is limited to the 20 minutes or 50 minutes session that the provider had with the patient, the provider is getting information from everyone who has touched the patient within the system of care and is therefore able to make a much better clinical decision. The provider has a better clinical picture of the patient and accordingly has an opportunity to provide better quality care. It may also help the provider document changes over time so they can modify their treatment regimen. An example of a graphical representation that can be generated at step 1108 is provided at FIG. 9.

FIG. 9 is a graph of diagnosis counts for various possible diagnoses over a period of time, according to an embodiment of the disclosure. The system can also generate a graph showing the trend of diagnosis counts over a period of time, such as the one shown in FIG. 9. The dates along the x-axis refer to dates of patient visits, and the numbers along the y-axis refer to diagnosis counts. To illustrate, at the patient's visit on Oct. 1, 2022, the narrative note had eight diagnosis indicators that could indicate depressive disorder, four that could indicate bipolar II disorder, three that could indicate bipolar II disorder (major depressive episode), and so on and so forth. An end user viewing this graph may come to the conclusion that the patient's depressive disorder is recently well managed, and now, generalized anxiety disorder is a bigger issue.

This graphical representation can provide a historical representation of all notes written for that patient over time by all provider types that have “touched” the patient. It can also be used by behavioral health providers and by medical providers in medical settings that would not be writing a behavioral health note. Unlike the convention situation where the provider's knowledge of the patient is limited to the 20 minutes or 50 minutes session that the provider had with the patient, the provider is getting information from everyone who has touched the patient within the system of care and is therefore able to make a much better clinical decision. The provider has a better clinical picture of the patient and accordingly has an opportunity to provide better quality care. It may also help the provider document changes over time so they can modify their treatment regimen.

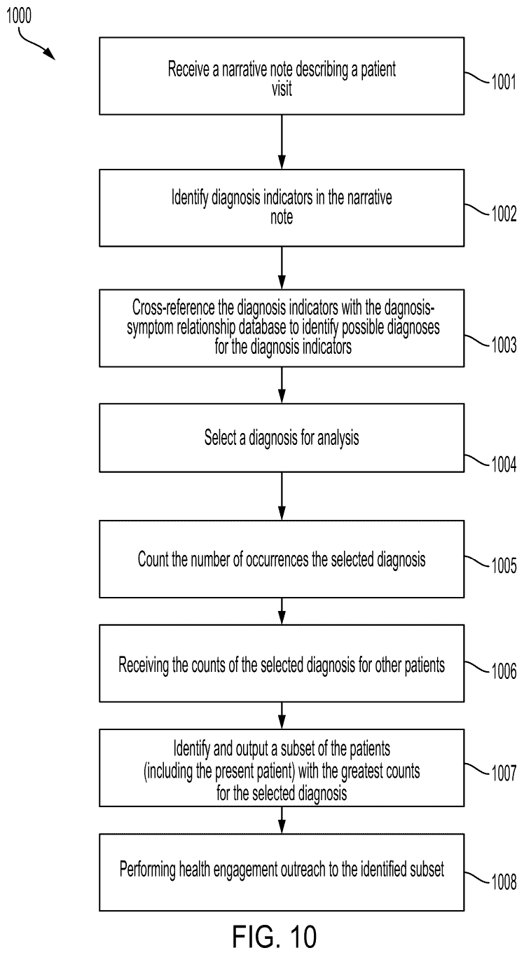

FIG. 10 is a flow chart of an exemplary method of diagnosis indicator identification and analytics for health engagement outreach. At step 1001, the method 1000 can include receiving a narrative note describing a patient visit. At step 1002, the method 1000 can include using the trained machine learning algorithm to identify diagnosis indicators in the narrative note. At step 1003, the method 1000 can include cross-referencing the diagnosis indicators identified at step 1002 with a master diagnosis-symptom relationship database to identify possible diagnosis for the diagnosis indicators.

At step 1004, the method 1000 can include selecting a possible diagnosis for analysis. Because organizations can use the results of this method to perform outreach with the hopes of saving money by getting patients in treatment, it may be desirable to select a diagnosis that is more chronic in nature and more costly, like schizophrenia or obsessive-compulsive disorder.

At step 1005, the method 1000 can include counting the number of occurrences for selected possible diagnosis. Said another way, the number of diagnosis indicators that could be indicative of the selected possible diagnosis are counted. At step 1006, the method 1000 can include receiving the counts for the selected diagnosis for other patients.

At step 1007, the method 1000 can include identifying and outputting a subset of the patients having the greatest counts. This subset represents a population group with the patients who are more “at risk” so an organization can target interventions for these patients first. At step 1008, the method 1000 can include performing health engagement outreach to that subset.

FIG. 11 is a flow chart of an exemplary method of diagnosis indicator identification and analytics for clinical decision support, according to an embodiment of the disclosure. At step 1101, the method 1100 can include receiving a narrative note describing a patient visit. At step 1102, the method 1100 can include using the trained machine learning algorithm to identify diagnosis indicators in the narrative note. At step 1103, the method 1100 can include selecting one of the diagnosis indicators for scoring.

At step 1104, the method 1100 can include scoring the selected diagnosis indicator based on its context in the narrative note. The scoring is performed using natural language processing, and is based on adjectives and adverbs describing a diagnosis indicator. For example, “client was severely depressed” will receive a higher score than “client exhibited signs of mild depression.” In some embodiments, scores can range from 0 to 1, with 0 being the least severe and 1 being the most severe.

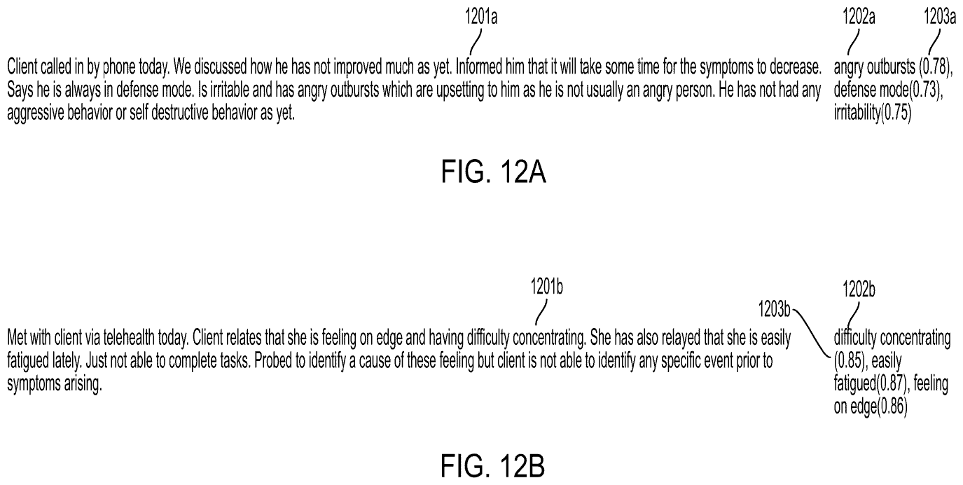

At step 1105, the method 1100 can include cross-referencing the selected diagnosis indicator with a severity database to identify a weight for the selected diagnosis indicator and then weighting the score calculated at step 1104 with that weight. While symptom severity may be subjective, there are certain symptoms that are known to be more severe than others, though they are not always more severe. For example, a patient may have suicidal ideation without a plan and also have homicidal ideation with a plan. The homicidal ideation with a plan would be scored higher than suicidal ideation without a plan. For another example, “suicidal” will be weighted higher than “stress.” In some embodiments, the severity database is periodically updated based on responses from patients, feedback from providers, and data analytics of system data. FIGS. 12A and 12B each show a narrative note 1201, the diagnosis indicators 1202 identified therein, and the associated scores 1203, according to embodiments of the disclosure.

At step 1106, the method 1100 can include receiving weighted scores for the selected diagnosis indicator from previous patient visits. These weighted scores may be from the same or different providers. For example, in some embodiments, the weighted scores may be from previous patient visits with a single provider. However, in some embodiments, the weighted scores may be from previous patient visits with many different providers.

At step 1107, the method 1100 can include trending the received and calculated weighted scores. At step 1108, the method 1100 can include generating and displaying a graphical representation of the trend from step 1107. The graphical representation can provide a historical representation of all notes written for that patient over time by all provider types that have “touched” the patient. It can also be used by behavioral health providers and by medical providers in medical settings that would not be writing a behavioral health note. Unlike the convention situation where the provider's knowledge of the patient is limited to the 20 minutes or 50 minutes session that the provider had with the patient, the provider is getting information from everyone who has touched the patient within the system of care and is therefore able to make a much better clinical decision. The provider has a better clinical picture of the patient and accordingly has an opportunity to provide better quality care. It may also help the provider document changes over time so they can modify their treatment regimen. An example of a graphical representation that can be generated at step 1108 is provided at FIG. 14.

To summarize, with this method 1100, each symptom expressed in a note can be scored at each session. Thus, each symptom could have a different score for each different session. The provider, in one view, can easily track progress to treatment, or lack thereof, and decide to continue the current treatment regimen or to refine it as symptoms change over time. For example, a patient has increased anxiety if his score increases from 0.080 to 0.085. Conversely, a patient has decreased anxiety if his score decreases from 0.80 to 0.75. In this way, the system displays historical data to inform the provider and assist in clinical decision making. Conventional solutions do not offer this is type of clinical decision support.

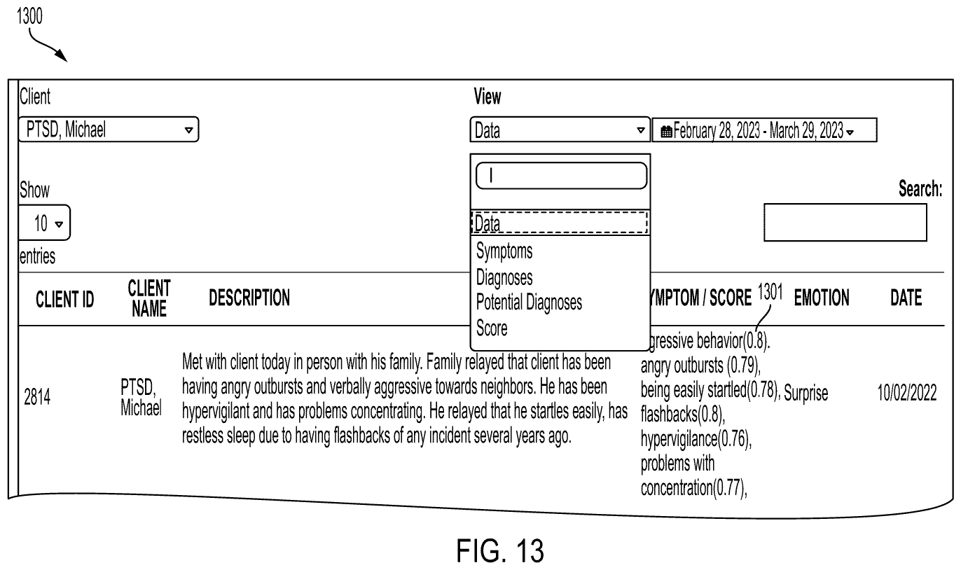

FIG. 13 illustrates the system's data module, according to an embodiment of the disclosure. The data module can also be referred to as the “symptom tracker module.” Users can access the symptom tracker module to view raw data, symptom trends, potential diagnosis, and scores 1301.

FIG. 14 is a graph of scores for a patient over a period of time, according to an embodiment of the disclosure. The system may display a graph similar to the one shown in FIG. 14 on an end user device. An end user may find this graph helpful for clinical decision support. For example, an end user viewing this graph may see that the patient was improving in November but worsening in December and January, which may prompt the end user to ask if anything in the patient's life changed between November and December that could have prompted this change. With that information, the end user may be able to more accurately diagnose and treat the patient.

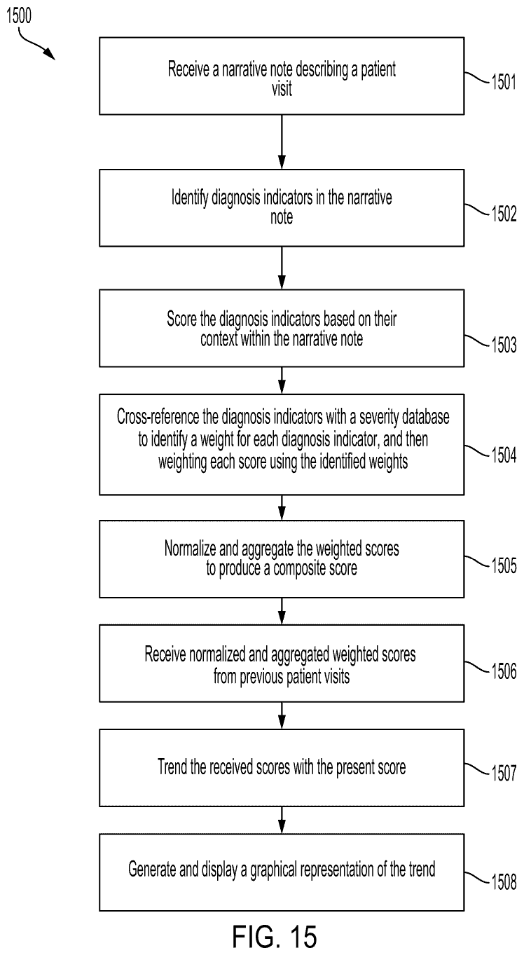

FIG. 15 is a flow chart of an exemplary method of diagnosis indicator identification and analytics for clinical decision support, according to an embodiment of the disclosure. At step 1501, the method 1500 can include receiving a narrative note describing a patient visit. At step 1502, the method 1500 can include using the trained machine learning algorithm to identify diagnosis indicators in the narrative note. At step 1504, the method 1500 can include scoring the diagnosis indicators identified at step 1502 based on their respective contexts in the narrative note. At step 1505, the method 1500 can include cross-referencing the diagnosis indicator identified at step 1502 with a severity database to identify a weight for reach diagnosis indicator, and then weighting the scores calculated at step 1504 with those weights.

At step 1506, the method 1500 can include normalizing and aggregating the weight scores to produce a composite score. If the same diagnosis indicator appears in a narrative note multiple times, a composite score is calculated for each occurrence and then the maximum is taken to proceed to step 1507.

Composite scores represent the severity of each symptom relative to the baseline period. The system creates normalized weighted scores using the following formula in some examples: x′=(x−xₘᵢₙ)/(xₘₐₓ−xₘᵢₙ). The goal of normalization in some examples is to change the values of numeric columns in the dataset to use a common scale, without distorting differences in the ranges of values or losing information. The normalized score can be scaled by multiplying the score by a scaling factor, which, in some embodiments, is 21.

A baseline score is created in some examples by taking an average of normalized scores from the first, e.g., three, patient visits. This baseline score is used to measure progress in each subsequent visit. Calculating the baseline score can include four steps: iterating over symptom data, normalization, aggregation, and aggregating baseline scores. At the iterating over symptom data step, the system iterates over each distinct encounter within the baseline period for a specific patient. For each encounter (e.g., visit), it calculates a weighted score for each symptom recorded during that encounter. At the normalization step, after calculating the weighted scores for each symptom, the system calculates the normalized score for each symptom within the encounter. This normalization is done by finding the minimum and maximum weighted scores across all symptoms within the encounter and scaling each symptom's weighted score accordingly. At the aggregation step, the normalized scores for each symptom within the encounter are then aggregated to calculate an average normalized score for the encounter. At the aggregating baseline scores step, the system aggregates the average normalized scores from all encounters within the baseline period to calculate the overall baseline score for the patient. This baseline score represents the average severity of symptoms experienced by the patient over the specified baseline period.

Put simply, the baseline score is the average of all the normalized weighted score within the base period (e.g., 3 days). The following formula is used to obtain the normalized value of each symptom for a day: x′=(x−xₘᵢₙ)/(xₘₐₓ−xₘᵢₙ). The average of all normalized scores of the day gives the composite score. By comparing the composite score of each day with the baseline score, one can see the changes as the treatment goes gradually day by day.

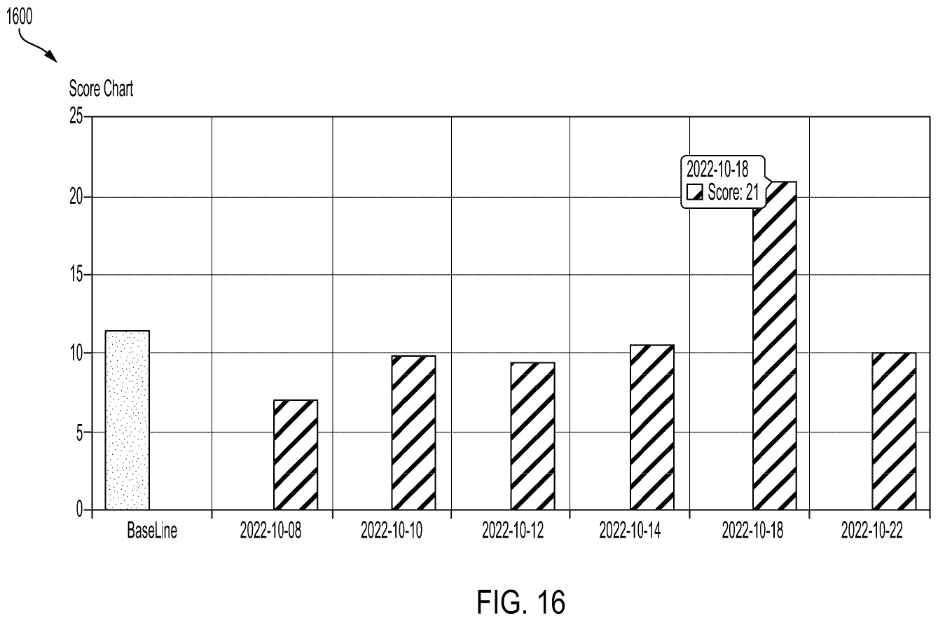

At step 1507, the method 1500 can include receiving normalized and aggregated weighted scores from previous patient visits. At step 1508, the method can include trending the received scores with the weighted score calculated at steps 1504-1506. At step 1509, the method can include generating and displaying a graphical representation of the trend. The graphical representation can provide a historical representation of all notes written for that patient over time by all provider types that have “touched” the patient. It can also be used by behavioral health providers and by medical providers in medical settings that would not be writing a behavioral health note. Unlike the convention situation where the provider's knowledge of the patient is limited to the 20 minutes or 50 minutes session that the provider had with the patient, the provider is getting information from everyone who has touched the patient within the system of care and is therefore able to make a much better clinical decision. The provider has a better clinical picture of the patient and accordingly has an opportunity to provide better quality care. An example of a graphical representation that can be generated at step 1509 is provided at FIG. 16.

FIG. 16 is a graph of composite scores for a patient over a period of time, according to an embodiment of the disclosure. The system may display a graph similar to the one shown in FIG. 16 on an end user device. An end user may find this graph helpful for clinical decision support. For example, an end user viewing this graph may see that the patient's behavioral health is relatively stable, except for on Nov. 18, 2022, in which the patient's score spiked, indicating poor behavioral health. Based on this graph, the end user may ask the patient what was going on in his life at that time. With that information, the end user may be able to more accurately diagnose and treat the patient.

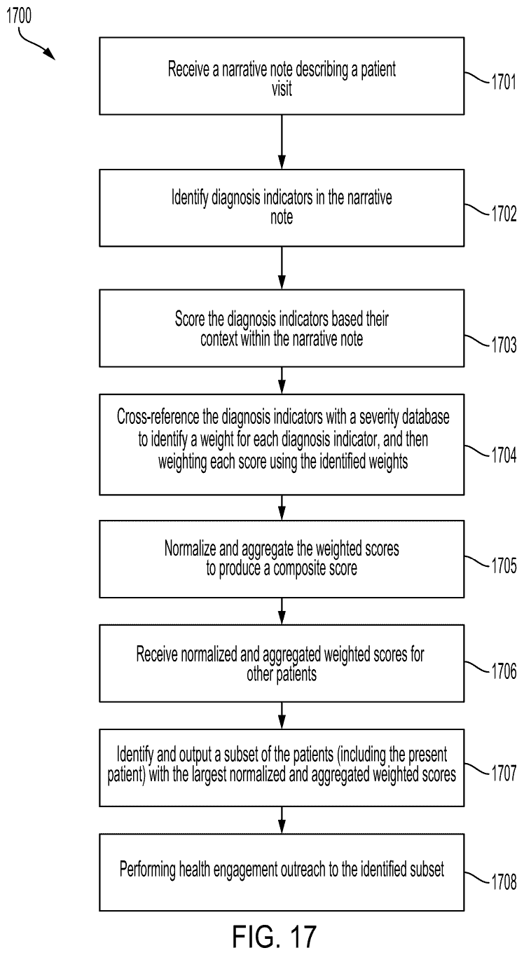

FIG. 17 is a flow chart of an exemplary method of diagnosis indicator identification and analytics for health engagement outreach, according to an embodiment of the disclosure. Steps 1701-1706 of method 1700 can be the same as steps 1501-1506 of method 1500. At step 1707, the method 1700 can include receiving normalized and aggregated weighted scores for other patients. At step 1708, the method 1700 can include identifying and outputting a subset of the patients with the largest normalized and aggregated weighted scores. At step 1709, the method 1700 can include performing health engagement outreach to that subset of patients. To provide an example, since the diagnosis indicator scoring is on a 0-1 scale, a patient with 5 or 6 symptoms with a composite score of say 3.2 would be more at risk than a patient that has 2 or 3 symptoms with a composite score of 1.2 and therefore be included in the subset of the patients with the largest normalized and aggregated weighted scores for outreach.

### Example Use Case

The following example use case describes an example of a typical user flow pattern. This section is intended solely for explanatory purposes and not in limitation.

The setting is point-of-care in a medical or behavioral health setting, first session. A clinician sees the patient. The clinician listens to the patient and then writes a note identifying the presenting problem, symptoms identified, their assessment of the patient at that time. Before finalizing the note that clinician would be rendered this graphic visualization based on the data they entered in their note shown in FIG. 5. The clinician may then change her evaluation of the patient based on the information provided and then complete an appropriate treatment plan based on the additional clinical decision support that was provided.

The clinician will advantageously see the screen illustrated in FIG. 7 first. Note that one symptom is associated with many diagnoses.

The system then renders the clustered rubric diagram shown in FIG. 8, which is digestible at the point of care before the clinician finalizes his/her note. This clustered rubric diagram makes it easy for the clinician to make his/her clinical decision. Most clinicians may focus on those boxes with a larger number of diagnosis matches. They can then ask some more pointed questions to get closer to the actual diagnosis.

The system then renders a graph similar to that shown in FIG. 9 to the clinician where the clinician can review all of the notes that were developed by all clinicians who have touched the patient over time.

With the graph shown in FIG. 9, in one easy-to-view graphic, the clinician can identify which diagnosis is trending more over time and also easily recognize changes in symptoms. Note the line associated with major depressive disorder starts high on the left and the line associated with generalized anxiety disorder has the highest score on the right of the graph. This indicates that the patient started out with depression but by this point in treatment their depression is well managed but their anxiety is high. This is now clearly evident to the clinician in a manner of seconds vs. hours of going through the chart. The clinician can now ask additional questions that will help them “treat to target” symptoms.

This same thinking can be applied on a population health level. On a population level, the goal is to review all patients across the system. This is where the scoring really comes in as the patients can now be stratified by “risk” based on the score, and an organization can take some action on the patients most in need.

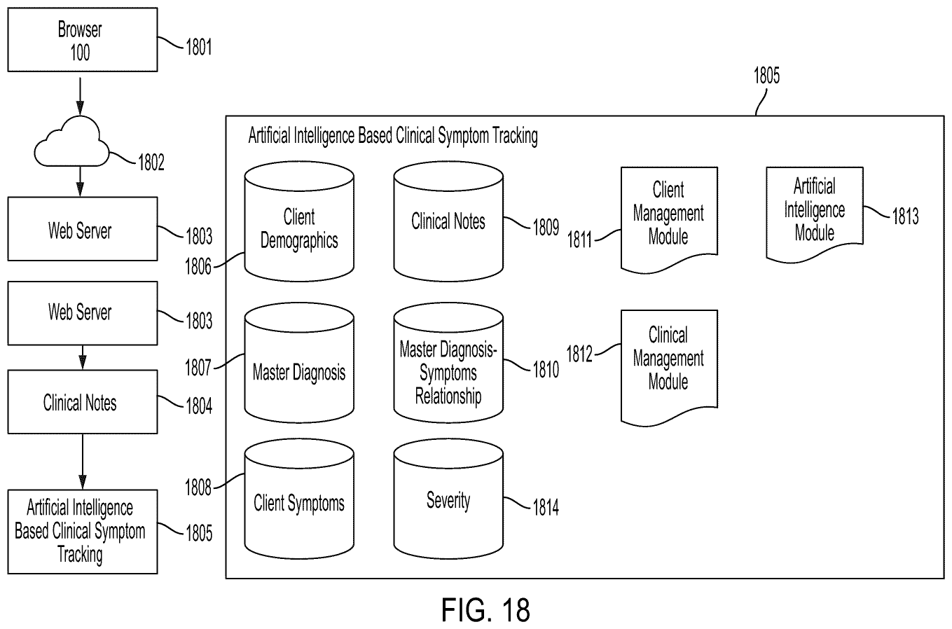

Returning to the figures, FIG. 18 illustrates an exemplary system for diagnosis indicator identification and analytics, according to an embodiment of the disclosure. An end user may enter a narrative note into the web browser 1801 that is then securely processed through the web browser and transmitted over the internet 1802 to the web server 1803 where the artificial intelligence based clinical symptom tracking module 1805 is hosted. Patient specific data can be pulled (1806). The system can then pull all clinical notes into a database (1809) and then feed the data to a client management module (1811) and then an artificial intelligence module (1813). Data may be stored in the modules to enhance the response and speed of the machine learning algorithm, which would bypass the need to pull the data fresh at each request. In some embodiments, the same process can extend to clinical notes (1804). The system can pull all diagnoses in the master diagnosis database (1807) and a master diagnosis-symptom relationship database (1810) to the clinical management module (1812). As the artificial intellectual clinical symptom tracking process (1805) continues to gather data it feeds it to the client symptoms database (1808) and to the severity database (1814). The artificial intelligence based clinical symptom tracking module 1805 allows clinicians to see the various symptoms and severity reflected in the narrative notes. All of the processing and pre-processing that is done accelerates the speed at which the data can be rendered back to the user.

In some embodiments, the system supports various kinds of clinical notes to support different types of services. Examples include DAP (Data, Assessment, Plan), SOAP (Subjective, Objective, Assessment and Plan), Psychiatrist Note, Medication Note etc.

The system may comprise several databases including a client demographics database 1806, a clinical notes database 1809, a master diagnosis database 1807, a master diagnosis-symptoms relationship database 1810, a client symptoms database 1808, and a severity database 1814. The client demographics database 1806 may store demographic information about the patient. This database maintains detailed demographic information about each individual receiving healthcare services. For example, the patient's age, gender, and marital status may be stored in the client demographics database 1806. Clinicians use this information to understand the individual's background during the clinical session.

The clinical notes database 1809 may store the narrative notes describing visits with the patient that are analyzed by the trained machine learning algorithm. The master diagnosis database 1807 may store a comprehensive list of possible diagnoses. This database can store a master list of diagnosis codes pertaining to behavioral health. In some embodiments, it can be populated in whole or in part with publicly available data sources, such as the Diagnostic and Statistical Manual of Mental Disorders and the International Statistical Classification of Diseases and Related Health Problems.

The master diagnosis-symptoms relationship database 1810 may store diagnoses and, for each diagnosis, the diagnoses indicators that may indicate the diagnosis. The client symptoms database 1808 may store diagnosis indicators identified for the patient. The severity database 1814 may store diagnosis indicators and associated weights.

In some embodiments, client records are stored in the master database, which could be an electronic health record, care management platform, health information exchange, etc. Once data has been sent to and processed by the system parts of the data, e.g., demographics, may be stored in system database in order to speed the response time to the user.

The system may also comprise several modules including a client management module 1811, an artificial intelligence module 1813, and a clinical management module 1812. In some embodiments, the client management module 1811 assists with managing new patients whose records are processed via the machine learning algorithm. In some embodiments, the artificial intelligence module 1813 can perform many steps of the methods described herein. In some embodiments, the clinical management module 1812 can provide other insights to the provider, e.g. a best practice for a particular diagnosis or an appropriate evidence-based practice for the diagnosis or recommendations based on their own dataset, such as, for example, “based on the response of 100 patient treated in our organization those with the best outcomes that had the same characteristics as this patient were treated by this ‘talk therapy,’ family intervention and X medication.”

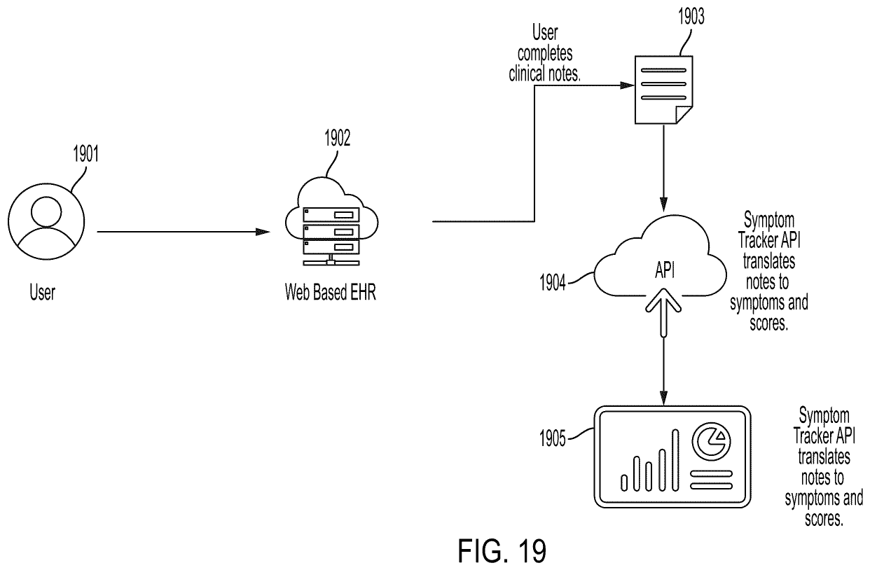

FIG. 19 illustrates a high-level flow diagram of exemplary system operation, according to an embodiment of the disclosure. As shown in FIG. 19, an end user 1901 uses a web browser 1902 to input a narrative note into the system 1903, and a trained machine learning algorithm translates the narrative note into diagnosis indicators (some of which are symptoms, as reference in the FIGS. 1904 and 1905.

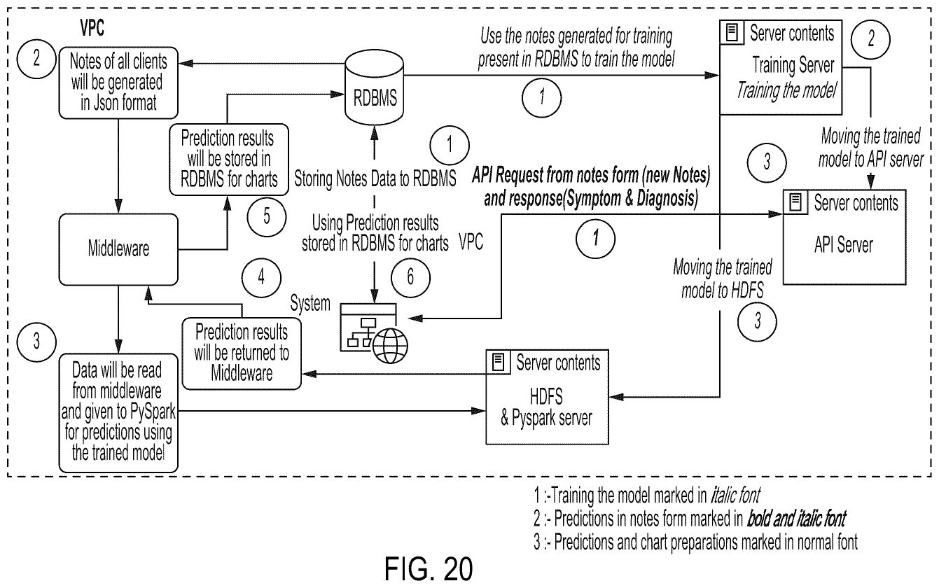

FIG. 20 illustrates a flow diagram of system operation, according to an embodiment of the disclosure. FIG. 20 shows three flow paths: training the machine learning algorithm (Flow 1), making predictions based on narrative notes (Flow 2), and making predictions and preparing charts (Flow 3). In some embodiments, Flow 1, training the machine learning algorithm, can include the following steps: using notes generated for training and present the notes in the relational database management system (RDBMS) in order to train the machine leaning model, training the machine learning model, and moving the trained machine learning model to an application programming interface (API) service, where predictions in notes are calculated and then moving the trained machine learning model to a Hadoop Distributed File System (HDFS), for example.

In some embodiments, during Flow 2, the machine learning algorithm makes predictions on the words or sentences that will describe a diagnosis indicator. These predications can be made when a new note comes in and can be processed in the master diagnosis-symptoms relationship database. In some embodiments, Flow 2 includes the following: An API request is made from the notes form (new notes) which returns a response with symptom and diagnosis.

Flow 3 describes the process for making the predictions and chart preparations. In some embodiments, in Flow 3, notes are stored from the notes form to the RDBMS. In some embodiments, the notes are generated in a JSON file type. The data is then read by middleware and will be used by one of the algorithms (for example, in some embodiments, PySpark) to predict symptoms using the trained machine learning algorithm. Predictions can then be stored as a JSON file and returned to Middleware. Prediction results can then be stored in the RDMS as symptoms with the associated diagnoses for displaying charts. The results of the stored charts can be displayed to the user from the RDMS upon electronic request from a user.

Making predictions and preparing charts can include the following steps: storing notes data to the RDBMS, generating notes of all patients, transferring the notes to middleware, reading data from middleware and transferring the data to the server hosting the machine learning algorithm to make predictions, returning the prediction results to middleware, storing the prediction results in the RDBMS for charts, and then using the prediction results stored in the RDBMS for charts.

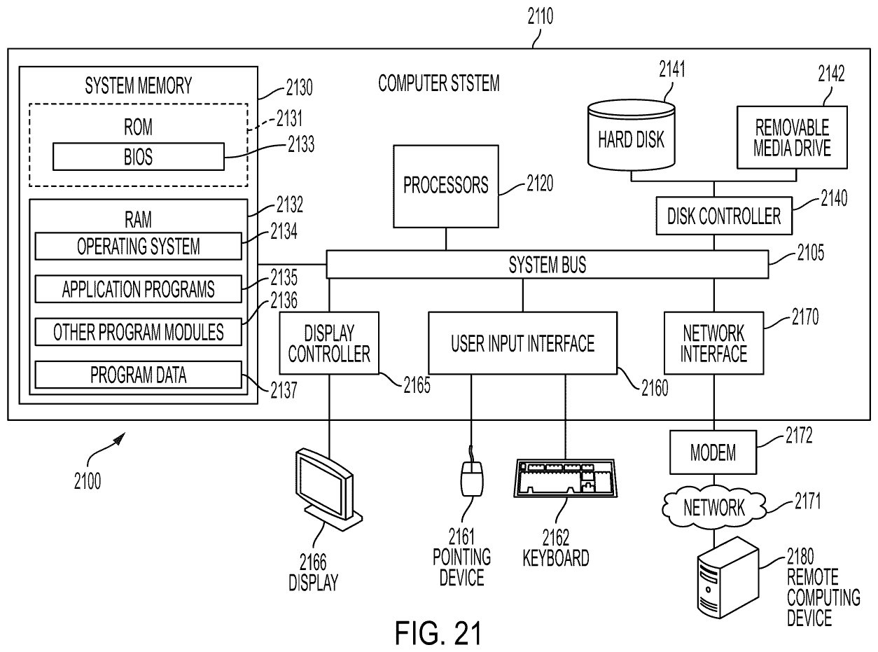

FIG. 21 illustrates an exemplary computing environment 2100 within which embodiments of the invention may be implemented. For example, this computing environment 2100 may be configured to execute a method of placing an item having irregular dimensions. The computing environment 2100 may include computer system 2110, which is one example of a computing system upon which embodiments of the invention may be implemented. Computers and computing environments, such as computer system 2110 and computing environment 2100, are known to those of skill in the art and thus are described briefly here.

As shown in FIG. 21, the computer system 2110 may include a communication mechanism such as a bus 2105 or other communication mechanism for communicating information within the computer system 2110. The computer system 2110 further includes one or more processors 2120 coupled with the bus 2105 for processing the information. The processors 2120 may include one or more central processing units (CPUs), graphical processing units (GPUs), or any other processor known in the art.

The computer system 2110 also includes a system memory 2130 coupled to the bus 2105 for storing information and instructions to be executed by processors 2120. The system memory 2130 may include computer readable storage media in the form of volatile and/or nonvolatile memory, such as read only memory (ROM) 2131 and/or random access memory (RAM) 2132. The system memory RAM 2132 may include other dynamic storage device(s) (e.g., dynamic RAM, static RAM, and synchronous DRAM). The system memory ROM 2131 may include other static storage device(s) (e.g., programmable ROM, erasable PROM, and electrically erasable PROM). In addition, the system memory 2130 may be used for storing temporary variables or other intermediate information during the execution of instructions by the processors 2120. A basic input/output system (BIOS) 2133 containing the basic routines that help to transfer information between elements within computer system 2110, such as during start-up, may be stored in ROM 2131. RAM 2132 may contain data and/or program modules that are immediately accessible to and/or presently being operated on by the processors 2120. System memory 2130 may additionally include, for example, operating system 2134, application programs 2135, other program modules 2136 and program data 2137.

The computer system 2110 also includes a disk controller 2140 coupled to the bus 2105 to control one or more storage devices for storing information and instructions, such as a hard disk 2141 and a removable media drive 2142 (e.g., floppy disk drive, compact disc drive, tape drive, and/or solid state drive). The storage devices may be added to the computer system 2110 using an appropriate device interface (e.g., a small computer system interface (SCSI), integrated device electronics (IDE), Universal Serial Bus (USB), or FireWire).

The computer system 2110 may also include a display controller 2165 coupled to the bus 2105 to control a display 2166, such as a cathode ray tube (CRT) or liquid crystal display (LCD), for displaying information to a computer user. The computer system 2110 includes an input interface 2160 and one or more input devices, such as a keyboard 2162 and a pointing device 2161, for interacting with a computer user and providing information to the processor 2120. The pointing device 2161, for example, may be a mouse, a trackball, or a pointing stick for communicating direction information and command selections to the processor 2120 and for controlling cursor movement on the display 2166. The display 2166 may provide a touch screen interface which allows input to supplement or replace the communication of direction information and command selections by the pointing device 2161.

The computer system 2110 may perform a portion or all of the processing steps of embodiments of the invention in response to the processors 2120 executing one or more sequences of one or more instructions contained in a memory, such as the system memory 2130. Such instructions may be read into the system memory 2130 from another computer readable medium, such as a hard disk 2141 or a removable media drive 2142. The hard disk 2141 may contain one or more datastores and data files used by embodiments of the present invention. Datastore contents and data files may be encrypted to improve security. The processors 2120 may also be employed in a multi-processing arrangement to execute the one or more sequences of instructions contained in system memory 2130. In alternative embodiments, hard-wired circuitry may be used in place of or in combination with software instructions. Thus, embodiments are not limited to any specific combination of hardware circuitry and software.

As stated above, the computer system 2110 may include at least one computer readable medium or memory for holding instructions programmed according to embodiments of the invention and for containing data structures, tables, records, or other data described herein. The term “computer readable medium” as used herein refers to any medium that participates in providing instructions to the processor 2120 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 2141 or removable media drive 2142. Non-limiting examples of volatile media include dynamic memory, such as system memory 2130. Non-limiting examples of transmission media include coaxial cables, copper wire, and fiber optics, including the wires that make up the bus 2105. Transmission media may also take the form of acoustic or light waves, such as those generated during radio wave and infrared data communications.

The computing environment 2100 may further include the computer system 2110 operating in a networked environment using logical connections to one or more remote computers, such as remote computer 2180. Remote computer 2180 may be a personal computer (laptop or desktop), a mobile device, a server, a router, a network PC, a peer device or other common network node, and typically includes many or all of the elements described above relative to computer system 2110. When used in a networking environment, computer system 2110 may include modem 2172 for establishing communications over a network 2171, such as the Internet. Modem 2172 may be connected to bus 2105 via user network interface 2170, or via another appropriate mechanism.

Network 2171 may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 2110 and other computers (e.g., remote computer 2180). The network 2171 may be wired, wireless or a combination thereof. Wired connections may be implemented using Ethernet, Universal Serial Bus (USB), RJ-11 or any other wired connection generally known in the art. Wireless connections may be implemented using Wi-Fi, WiMAX, and Bluetooth, infrared, cellular networks, satellite or any other wireless connection methodology generally known in the art. Additionally, several networks may work alone or in communication with each other to facilitate communication in the network 2171.

The embodiments of the present disclosure may be implemented with any combination of hardware and software. In addition, the embodiments of the present disclosure may be included in an article of manufacture (e.g., one or more computer program products) having, for example, computer-readable, non-transitory media. The media has embodied therein, for instance, computer readable program code for providing and facilitating the mechanisms of the embodiments of the present disclosure. The article of manufacture can be included as part of a computer system or sold separately.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A graphical user interface (GUI), as used herein, comprises one or more display images, generated by a display processor and enabling user interaction with a processor or other device and associated data acquisition and processing functions. The GUI also includes an executable procedure or executable application. The executable procedure or executable application conditions the display processor to generate signals representing the GUI display images. These signals are supplied to a display device which displays the image for viewing by the user. The processor, under control of an executable procedure or executable application, manipulates the GUI display images in response to signals received from the input devices. In this way, the user may interact with the display image using the input devices, enabling user interaction with the processor or other device.

The functions and process steps herein may be performed automatically or wholly or partially in response to user command. An activity (including a step) performed automatically is performed in response to one or more executable instructions or device operation without user direct initiation of the activity.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

Aspects of the present technical solutions are described herein with reference to flowchart illustrations and/or block diagrams of methods, apparatuses (systems), and computer program products according to embodiments of the technical solutions. It will be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present technical solutions. In this regard, each block in the flowchart or block diagrams can represent a module, segment, or portion of instructions, which includes one or more executable instructions for implementing the specified logical function(s). In some alternative implementations, the functions noted in the blocks can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

A second action can be said to be “in response to” a first action independent of whether the second action results directly or indirectly from the first action. The second action can occur at a substantially later time than the first action and still be in response to the first action. Similarly, the second action can be said to be in response to the first action even if intervening actions take place between the first action and the second action, and even if one or more of the intervening actions directly cause the second action to be performed. For example, a second action can be in response to a first action if the first action sets a flag and a third action later initiates the second action whenever the flag is set.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A computer implemented method for diagnosis indicator identification and analytics in a data processing system comprising a processing device and a memory comprising instructions which are executed by the processing device, the method comprising:
receiving, from an end user device via one or more networks, a narrative note describing a first patient visit with a first patient, wherein the narrative note comprises unstructured data;
applying a machine learning algorithm to the unstructured data of the narrative note to identify a plurality of diagnosis indicators, wherein the machine learning model is trained at least in part to use natural language processing to identify the plurality of diagnosis indicators;
performing at least one analysis based on information regarding the plurality of diagnosis indicators to thereby identify at least one possible patient diagnosis; and
outputting a visual representation of results of the at least one analysis via the one or more networks and to the end user device for display on a display device of the end user device, wherein the result is associated with the at least one possible patient diagnosis and the visual representation facilitates one or more of clinical decision making and health engagement outreach.

2. The computer implemented method of claim 1, wherein the plurality of diagnosis indicators comprise one or more of a symptom or a phrase, wherein the phrase describes one of a personal experience or an external experience of the first patient.

3. The computer implemented method of claim 1, further comprising:
providing, to the end user device via the one or more networks, the narrative note and the plurality of diagnosis indicators;
obtaining, from the end user device, at least one indication that at least one of the plurality of diagnosis indicators are one of correct or incorrect; and
retraining the machine learning algorithm based on the at least one indication.

4. The computer implemented method of claim 3, further comprising repeating the receiving, applying, performing, outputting, providing, obtaining, and retraining until an accuracy threshold for the machine learning algorithm is exceeded.

5. The computer implemented method of claim 4, further comprising deploying the machine learning algorithm on an application programming interface (API) server such that the machine learning algorithm is accessible via a provided API to a plurality of end user devices via another one or more networks.

6. The computer implemented method of claim 1, wherein the at least one analysis comprises:
cross-referencing each of the plurality of diagnosis indicators with a diagnosis-symptom database comprising diagnoses and diagnosis indicators associated with each diagnosis;
identifying, for each of the plurality of diagnosis indicators, the at least one possible patient diagnosis based on the cross-referencing; and
counting a number of occurrences for each of the at least one possible patient diagnosis.

7. The computer implemented method of claim 6, wherein subsets of identified possible patient diagnoses with similar numbers of occurrences are clustered together in the visual representation to thereby yield insights from clusters to improve clinical decision making.

8. The computer implemented method of claim 6, further comprising:
receiving, for each of a plurality of other patients, the number of occurrences for the at least one possible patient diagnosis; and
identifying a subset of the first patient and the plurality of other patients associated with the greatest counts for the at least one possible patient diagnosis, wherein the visual representation comprises identifiers for patients in the subset to facilitate health engagement outreach to the patients in the subset.

9. The computer implemented method of claim 1, wherein the at least one analysis comprises:
selecting one of the plurality of diagnosis indicators for analysis;
calculating a first score for the selected diagnosis indicator based on context within the narrative note;
receiving a plurality of previous scores associated with the selected diagnosis indicator and associated with a plurality of previous patient visits; and
trending the plurality of previous scores and the first score for the selected diagnosis indicator, wherein the visual representation comprises a graphical representation resulting from the trending that facilitates clinical decision making.

10. The computer implemented method of claim 9, wherein context comprises one or more of an adjective or an adverb qualifying the selected diagnosis indicator.

11. The computer implemented method of claim 10, wherein a first diagnosis indicator qualified by the adjective “severe” is scored higher than a second diagnosis indicator qualified by the adjective “mild.”

12. The computer implemented method of claim 9, wherein the plurality of previous scores is received from a plurality of end user devices, each associated with a different health care provider for the first patient.

13. The computer implemented method of claim 9, further comprising:
cross-referencing the selected diagnosis indicator with a severity database;
identifying a weight based on the cross-referencing; and
calculating a weighted score by multiplying the first score for the selected diagnosis indicator by the weight.

14. The computer implemented method of claim 1, wherein the at least one analysis comprises:
scoring each of the plurality of diagnosis indicators based on a respective context within the narrative note;
cross-referencing each of the plurality of diagnosis indicators with a severity database;
identifying, for each of the plurality of diagnosis indicators, a respective weight based on the cross-referencing; and
calculating, for each of the plurality of diagnosis indicators, a respective weighted score by multiplying a respective score for the plurality of diagnosis indicators by a respective weight.

15. The computer implemented method of claim 14, wherein the at least one analysis comprises:
normalizing and aggregating the weighted scores to produce a first composite score;
receiving, a plurality of previous composite scores; and
trending the plurality of previous composite scores and the first composite score, wherein the visual representation comprises a graphical representation of a trend resulting from the trending that facilitates clinical decision making.

16. The computer implemented method of claim 1, further comprising:
scoring each of the plurality of diagnosis indicators based on a respective context within the narrative note;
cross-referencing each of the plurality of diagnosis indicators with a severity database;
identifying, for each of the plurality of diagnosis indicators, a respective weight based on the cross-referencing;
calculating, for each of the plurality of diagnosis indicators, a respective weighted score by multiplying a respective score for each of the plurality of diagnosis indicators by a respective weight;
normalizing and aggregating the weighted scores to produce a first composite score;
receiving, for each of a plurality of other patients, a respective composite score; and
identifying a subset of the first patient and the plurality of other patients associated with the greatest composite scores, wherein the visual representation comprises names of patients in the subset to assist a user of the end user device in performing health engagement outreach to the patients in the subset.

17. A computer program product for diagnosis indicator identification and analytics, the computer program product comprising a non-transitory computer readable storage medium having program instructions embodied therewith, the program instructions executable by a processor to cause the processor to:
receive, from an end user device via one or more network and an application programming interface (API) provided to the end user device, unstructured data comprising a narrative note describing a first patient visit with a first patient;
identify, using a machine learning algorithm trained to use one or more natural language processing algorithms, a plurality diagnosis indicators in the unstructured data;
perform at least one analysis based on obtained information regarding the plurality of diagnosis indicators; and
output via the one or more networks a visual representation of a result of the at least one analysis to the end user device for display.

18. The computer program product of claim 17, wherein the at least one analysis comprises:
cross-referencing each of the plurality of diagnosis indicators with a diagnosis-symptom database comprising diagnoses and diagnosis indicators associated with each diagnosis;
identifying, for each of the plurality of diagnosis indicators, at least one possible patient diagnosis based on the cross-referencing; and
counting a number of occurrences for each of the at least one possible patient diagnosis, wherein the visual representation comprises a graphical representation of the at least one possible patient diagnosis with subsets of possible patient diagnoses with similar numbers of occurrences clustered together in the graphical representation, wherein a user of the end user device can glean insights from clusters to improve clinical decision making.

19. The computer program product of claim 17, wherein the at least one analysis comprises:
selecting one of the plurality of diagnosis indicators for analysis;
calculating a first score for the selected diagnosis indicator based on context within the narrative note;
cross-referencing the selected diagnosis indicator with a severity database;
identifying a weight based on the cross-referencing;
calculating a first weighted score by multiplying the first score for the selected diagnosis indicator by the weight;
receiving, from previous patient visits from a plurality of end user devices, each associated with a different health care provider for the first patient, a plurality of previous scores associated with the selected diagnosis indicator; and
trending the plurality of previous scores and the first weighted score, wherein the visual representation comprises a graphical representation of the trend that assists a user of the end user device in clinical decision making.

20. A system for diagnosis indicator identification comprising a processing device and a memory comprising instructions that when executed by the processing device cause the system to:
train a machine learning model to identify diagnosis indicators;
receive, from an end user device via one or more networks, a narrative note comprising unstructured data describing a first patient visit with a first patient;
identify, using the machine learning model, a plurality of diagnosis indicators in the narrative note, wherein the machine learning model is trained at least in part to use one or more natural language processing algorithms to analyze the unstructured data;
output, to the end user device via the one or more networks, the narrative note and the plurality of diagnosis indicators;
receive, from the end user device, at least one indication that at least one of the plurality of diagnosis indicators are one of correct or incorrect;
retrain the machine learning model based on the at least one indication;
determine that an accuracy threshold is exceeded for the machine learning model as a result of the retraining; and
deploy the machine learning model on an application programming interface (API) server such that the machine learning model is accessible via a provided API to a plurality of end user devices via another one or more networks.
